(19) United States
(12) Patent Application Publication
(10) Pub. No.: US 2024/0140989 A1
(43) Pub. Date: May 2, 2024

(54) A METHOD AND SYSTEM FOR INTEGRATED AND CONTINUOUS VIRAL FILTRATION, CONCENTRATION AND BUFFER EXCHANGE

(71) Applicant: Boehringer Ingelheim International GmbH, Ingelheim am Rhein (DE)

(72) Inventors: Charles CAPRON, Oakland, CA (US); Scott GODFREY, Pleasanton, CA (US); Nikita IKON, Union City (UA); Joelle KHOURI, Oakland, CA (US); Raquel OROZCO, Richmond, CA (US); Min ZHU, Union City, CA (US)

(21) Appl. No.: 18/561,823

(22) PCT Filed: May 24, 2022
(86) PCT No.: PCT/US2022/030783

§ 371 Date: Nov. 17, 2023

Related U.S. Application Data
(60) Provisional application No. 63/192,662, filed on May 25, 2021.

(51) Int. Cl. C07K 1/34

(52) U.S. Cl. CPC C07K 1/34

(57) ABSTRACT

A method and system for integrated and continuous viral filtration and biological product concentration, including an initial purification system coupled to a final purification system. The initial purification system includes a viral reduction filtration (VRF) skid, while the final purification system includes a single-pass tangential filtration-diafiltration (SPTFF-DF) skid.

### TECHNICAL FIELD

Disclosed herein are systems and methods for use in the manufacturing of biologic products (e.g. proteins), and more particularly a single-use system and method for integrated and continuous viral filtration, concentration and buffer exchange. In certain embodiments, the system is an integrated, single-use system containing a viral filtration unit operation coupled to a single-pass tangential flow filtration (SPTFF) and diafiltration (DF) unit operation for use in processing a feed stream comprising a biologic product or material of interest.

### BACKGROUND

A significant challenge in the manufacturing of any therapeutic agent is the ability to provide consistently efficacious product of the required purity, free from environmental and process-related contamination. The manufacture of biologics is particularly challenging, given the involvement of living cells.

Traditional biologics manufacturing consists of a similar sequence of unit operations that are divided into two main parts: upstream and downstream. The upstream unit operations typically include cell culture and harvest steps, while the downstream consists of multiple purification steps. Specifically, the end of the downstream process usually includes a viral reduction filtration (VRF or VF) step, followed by ultrafiltration/diafiltration (UF/DF) for product concentration and buffer exchange.

Conventionally, VRF and UF/DF systems are decoupled and carried out sequentially, often on separate days, and utilize large equipment that takes up significant space. While new technologies seek to reduce the process time of VRF systems via periodic/continuous filtration over the course of a production run, there remain concerns regarding viral breakthrough.

There is a need in the art for improved processes and, in particular, VRF and UF/DF processes that permit a smaller footprint for large-scale production, in a rapid timeframe, using single-use equipment, while minimizing the risk of viral breakthrough.

### SUMMARY OF THE INVENTION

Disclosed herein are systems and methods for integrated, continuous viral filtration, ultrafiltration and diafiltration for use in the manufacture of a biologic product, such as a monoclonal antibody, and in particular, in the processing of a feed stream produced by batch or continuous manufacturing of a biologic product of interest.

In one aspect, a single-use system for integrated, continuous processing of an initial biologic product is provided, wherein the system comprises a viral filtration unit operation coupled to a single-pass tangential flow filtration (SPTFF) and diafiltration (DF) unit operation.

In another aspect, an integrated, continuous method for providing a processed biologic product is provided, the method comprising a) providing a feed stream (e.g. a fluid feed) comprising an initial biologic product; b) filtering the feed stream to remove viral contaminants; c) concentrating the initial biologic product; and d) conducting buffer exchange to produce a processed biologic product.

In a third aspect, a method of manufacturing a biologic product of interest is provided, the method comprising the steps of:
(I) cultivating a eukaryotic cell expressing the biologic product of interest in cell culture;
(II) harvesting the biologic product of interest from the cell culture in the form of a fluid feed comprising biologic product of interest and one or more impurities or buffer components;
(III) purifying the fluid feed comprising biologic product of interest and one or more impurities or buffer components to isolate the biologic product of interest from the fluid feed; and
(IV) optionally formulating the biologic product of interest into a pharmaceutically acceptable formulation suitable for administration; and wherein the method further comprises the step of: passing the fluid feed through a single-use system for integrated, continuous processing of an initial biologic product;
wherein the single-use system for integrated, continuous processing of an initial biologic product comprises a viral filtration unit operation coupled to a single-pass tangential flow filtration (SPTFF) and diafiltration (DF) unit operation.

In a fourth aspect, a method of manufacturing a biologic product of interest is provided, the method comprising the steps of:
(I) cultivating a eukaryotic cell expressing the biologic product of interest in cell culture;
(II) harvesting the biologic product of interest from the cell culture in the form of a fluid feed comprising biologic product of interest and one or more impurities or buffer components;
(III) purifying the fluid feed comprising biologic product of interest and one or more impurities or buffer components to isolate the biologic product of interest from the fluid feed; and
(IV) optionally formulating the biologic product of interest into a pharmaceutically acceptable formulation suitable for administration; and
wherein the method further comprises:
a) providing a feed stream (e.g. a fluid feed) comprising an initial biologic product; b) filtering the feed stream to remove viral contaminants; c) concentrating the initial biologic product; and d) conducting buffer exchange to produce a processed biologic product.

In a fifth aspect, a viral filtration-ultrafiltration and diafiltration (VF-UPDF) system is disclosed that includes an initial purification unit operation and a final purification unit operation, wherein the unit operations are coupled.

In one embodiment, the initial purification unit operation contains at least one viral filtration membrane for removing viral particles and the final purification unit operation contains a single-pass tangential flow filtration (SPTFF) and diafiltration (DF) system for concentration and buffer exchange.

In one embodiment, the initial purification unit operation contains a pump, at least one pre-filter and one or more viral reduction filtration membranes.

In one embodiment, the final purification unit operation contains one or more SPTFF membranes, DF mixing tanks, DF membranes, sensors, pumps or a combination thereof.

In one embodiment, the material is a protein.

In a particular embodiment, the material is a monoclonal antibody.

In one embodiment, the processing is conducted within a time frame reduced by about 50% compared to a conventional processing system.

In one embodiment, the processing is conducted within a period of about 24 hours or less.

In one embodiment, the processing is conducted within a period of about 12 hours or less.

In one embodiment, the processing results in a ten-fold increase in concentration of the material.

In one embodiment, the system further comprises a feed reservoir coupled to the initial purification assembly.

In a particular embodiment, the feed reservoir holds purified and polished monoclonal antibody at a concentration between about 5 and about 20 g/L or more particularly, between about 8 about 12 g/L.

### BRIEF DESCRIPTION OF THE DRAWINGS

The foregoing and other features and aspects of the invention may be best understood with reference to the following description of certain exemplary embodiments of the invention, when read in conjunction with the accompanying drawings, wherein:

FIG. 1 shows a schematic of an integrated single-use system for performing a final purification process that includes viral filtration (VF), as the last step in the initial purification suite, coupled to a single-pass tangential flow filtration (SPTFF) and diafiltration (DF) system for concentration and buffer exchange in the final purification suite, in accordance with an exemplary embodiment.

FIG. 2 shows a schematic of the viral filtration system used within the integrated single-use system of FIG. 1 in accordance with an exemplary embodiment.

FIG. 3 shows a schematic of the final purification system used within the integrated single-use system of FIG. 1 in accordance with an exemplary embodiment.

FIG. 4 shows a Mode 1 and 2 pictorial time representation comparing the operating times of the integrated single-use system when operating in Mode 1 or Mode 2 in accordance with an exemplary embodiment.

FIG. 5A shows a graph of a Volumetric Conversion Factor Flux Excursion Profile for a Pall 4-In-Series (Molecule 1) which illustrates the volumetric conversion factor (VCF) versus feed flux for Molecule 1 at various starting concentrations using a Pall 4-in-series membrane for the SPTFF membrane in accordance with an exemplary embodiment.

FIG. 5B shows a graph of a Volumetric Conversion Factor Flux Excursion Profile for a Pall 9-In-Series (Molecule 2) which illustrates the volumetric conversion factor (VCF) versus feed flux for Molecule 2 at various starting concentrations using a Pall 9-in-series membrane for the SPTFF membrane in accordance with an exemplary embodiment.

FIG. 5C shows a graph of a Volumetric Conversion Factor Flux Excursion Profile for a Pall 9-In-Series (Molecule 3) which illustrates the volumetric conversion factor (VCF) versus feed flux for Molecule 3 at various starting concentrations using a Pall 9-in-series membrane for the SPTFF membrane in accordance with an exemplary embodiment.

The drawings illustrate only exemplary embodiments of the invention and are therefore not to be considered limiting of its scope, as the invention may admit to other equally effective embodiments.

### DETAILED DESCRIPTION

To produce a biologic product from a fluid (e.g., cell culture media or clarified cell culture media), purification and/or particle separation may be necessary. In conventional approaches, the process of purifying a fluid and/or separating particles (i.e., solids) from the fluid includes a number of steps, each of which is carried out in a separate equipment system. The cost of the process typically includes separate equipment and space costs for control and processing hardware associated with each process step.

Disclosed herein are methods, systems and apparatuses used in filtration processes of a molecule, such as a protein for example, and more particularly a method and system for integrated and continuous viral filtration, ultrafiltration, and diafiltration processes of a molecule. Advantageously, the systems and methods disclosed herein permit viral filtration, ultrafiltration and diafiltration in a compact format, i.e., with a smaller footprint, than conventional approaches.

Although the description of exemplary embodiments of the invention are provided below in conjunction with utilizing certain particular equipment, alternative embodiments of the invention may be applicable to other types of equipment used within the process that perform the same or similar function and are collectively space-saving when compared to traditional systems.

### I. Definitions

The term “biologic product” or “biologic material” generally refers to a product of interest created via biological processes or via the chemical or catalytic modification of an existing biologic product. Biological processes include cell culture, fermentation, metabolization, respiration, and the like. Biologic products of interest include, for example, antibodies, antibody fragments, proteins, hormones, vaccines, fragments of natural proteins (such as fragments of bacterial toxins used as vaccines, e.g., tetanus toxoid), fusion proteins or peptide conjugates (e.g., such as subunit vaccines), virus-like particles (VLPs) and the like.

The term “continuous” as used herein refers to two or more integrated (physically connected) continuous unit operations with minimal hold volume in between. Such processes are also referred to as fully continuous or end-to-end continuous. A process is hybrid if it is composed of both batch and continuous unit operations, e.g., a continuous upstream process (cell culture and synthesis of the target protein) and a batch downstream (purification and formulation of the protein into a drug substance or drug product). In the specific context of linked unit operations described herein, the term “continuous” refers to a constant or non-periodic liquid transfer. In one embodiment, the methods and systems described herein permit continuous viral filtration, concentration and buffer exchange with respect to a batch of protein.

The term “diafiltration” or “DF” is used to mean buffer exchange, i.e., the replacement of one set of buffer salts with another set.

The term “diavolume” or “DV” is a measure of the extent of washing that has been performed during a diafiltration step. It is based on the volume of diafiltration buffer introduced into the unit operation compared to the retentate volume.

The terms “downstream” or “downstream processing” generally refer to some or all of the steps necessary for capture of a biologic product from the original solution in which it was created, for purification of the biologic product away from undesired components and impurities, for filtration or deactivation of pathogens (e.g., viruses, endotoxins), and for formulation and packaging.

The term “highly concentrated” refers to a concentration which is higher than the starting concentration, preferably significantly higher than before. The amount of increase of the concentration depends upon, for example, the biomolecule and medium chosen as well as conditions and parameters of the ultrafiltration and diafiltration equipment used. In certain embodiments described herein, the final protein concentration is between about 1 and about 80 g/L, about 10 and about 80 g/L, about 20 and about 80 g/L, about 20 and about 70 g/L, about 30 and about 70 g/L, or more particularly, about 1 and about 10 g/L, about 10 and about 20 g/L, about 20 and about 30 g/L, about 30 and about 40 g/L, about 40 and about 50 g/L, about 50 and about 60 g/L, about 60 and about 70 g/L, about 70 and about 80 g/L. In certain embodiments, the final protein concentration is greater than about 80 g/L. In certain embodiments, the final protein concentration is increased two-fold, three-fold, four-fold, five-fold or ten-fold or more over the concentration of protein in the feed. In a particular embodiment, 10 g/L of material in 100 L is brought to 100 g/L in 10 L, i.e., a ten-fold increase in concentration.

The term “feed”, “feed sample” and “feed stream” refer to the solution that is delivered (e.g., continuously, as a batch) to a unit operation (e.g. viral filtration, SPTFF) to be filtered.

The term “filtration” as used herein refers to a pressure-driven separation process that uses membranes to separate components in a liquid solution or suspension according to size differences between the components. Filtering results in the removal of at least part of (e.g., at least 80%, 90%, 95%, 96%, 97%, 98%, or 99%) undesired biological contaminants (e.g., a mammalian cell, bacteria, yeast cells, viruses, or mycobacteria) and/or particulate matter (e.g., precipitated proteins) from a liquid (e.g., a liquid culture medium or fluid present in any of the systems or processes described herein).

The term “filtrate” as used herein means a fluid that is emitted from a filter (e.g., a pre-filter, or a virus filter) that includes a detectable amount of a recombinant antibody.

The term “flow path” as used herein refers to a channel supporting the flow of a liquid (e.g., feed, retentate, permeate) through all or part of a system or sub-system.

The term “integrated” with respect to a system or process herein means a system or process in which structural elements function cooperatively to achieve a specific result (e.g., the generation of a monoclonal antibody from a liquid culture medium).

The term “microfiltration” refers to filtration used to separate intact cells and relatively large debris or protein aggregates from a mixture utilizing pore sizes in the range of about 0.05 μm to about 1 μm in diameter.

The term “perfusion cell culture” as used herein refers to perfusion cultivation which is carried out by continuously feeding fresh medium to the bioreactor and constantly removing the cell-free spent medium while retaining the cells in the reactor; thus, a higher cell density can be obtained in perfusion cultures compared to continuous cultures, as cells are retained within the reactor via a cell retention device. The perfusion rate depends on the demand of cell line, the concentration of nutrients in the feed and the level of toxification.

The terms “polypeptide”, “polypeptide product”, “protein” and “protein product”, are used interchangeably herein and, as is known in the art, refer to a molecule consisting of two or more amino acids, e.g., at least one chain of amino acids linked via sequential peptide bonds. In one embodiment, a “protein of interest” or a “polypeptide of interest” is a protein encoded by an exogenous nucleic acid molecule that has been transformed into a host cell, wherein the exogenous DNA determines the sequence of amino acids. In another embodiment, a “protein of interest” is a protein encoded by a nucleic acid molecule that is endogenous to the host cell.

The term “pre-filter” as used herein refers to a filter upstream of a viral filtration membrane. The purpose of the pre-filter is to selectively retain plugging constituents before the viral reduction filtration step while permitting passage of the biologic product of interest.

The term “retention” as used herein refers to the fraction of a particular biologic product (e.g., protein) that is retained by the membrane. It can also be calculated as either apparent or intrinsic retention.

The term “single-use” as used herein refers to articles that are suitable for one-time use with subsequent disposal, as well as reusable articles which are used only once in the process according to the invention and are then no longer used in the process. Such articles can also be referred to as “disposable”.

The term “skid” refers to a system of components contained within a frame that allows the system to be easily transported. Individual skids can contain complete process systems or systems that carry out certain aspects of a process. Multiple skids can be combined to create larger systems or entire portable plants.

The term “single-pass tangential flow filtration” or “SPTFF” is a type of tangential flow filtration where the feed flow is directed through the filter device in a single pass without recirculation.

The term “tangential flow filtration” or “TFF”, also known as crossflow filtration, refers to a process where the feed stream flows parallel to the membrane face. Applied pressure causes one portion of the flow stream to pass through the membrane (filtrate/permeate) while the rest of the flow stream (retentate) is retained. In traditional TFF, the retentate is recirculated back to the feed reservoir.

The term “transmembrane pressure” or “TMP” refers to the average applied pressure from the feed to the filtrate side of a membrane.

The term “ultrafiltration” or “UF” as used herein refers to any technique in which a solution or a suspension is subjected to a semi-permeable membrane that retains macromolecules while allowing solvent and small solute molecules to pass through. Ultrafiltration may be used to increase the concentration of macromolecules in a solution or suspension. In an embodiment, ultrafiltration is used to increase the concentration of a protein in water. Membrane ratings may be expressed in nominal molecular weight (NMW) and in the range of about 1 kD to about 1000 kD, for example.

The term “unit operation” refers to a functional step that can be performed in a process of manufacturing a biologic substance from a liquid culture medium.

The term “viral reduction filtration” or “VRF” or “VF” refers to a common unit operation in biomanufacturing intended to reduce viral contamination. The process retains virus particles on the filter's surface and within the pores and is based on virus size. Viral filters can be located at various points in a typical protein purification process. In one embodiment, the viral filter is located immediately upstream of a UF/DF. Viral reduction levels are calculated by comparing the amount of virus in a preprocessed load material to that in a postprocessed sample. The level is typically expressed in terms of the logarithm (log 10) of the reduction. Virus clearance filters are broadly classified into two categories: filters that provide >4 or >6 log 10 removal of large viruses, typically 80-100 nm endogenous retroviruses; and filters that provide >4 log 10 removal of small and large viruses (larger than 18-24 nm parvoviruses). The decrease in the number of viral particles can be on the order of about 1% to about 99%, preferably of about 20% to about 99%, more preferably of about 30% to about 99%, more preferably of about 40% to about 99%, even more preferably of about 50% to about 99%, even more preferably of about 60% to about 99%, yet more preferably of about 70% to about 99%, yet more preferably of about 80% to 99%, and yet more preferably of about 90% to about 99%. In certain non-limiting embodiments, the amount of virus, if any, in the purified antibody product is less than the ID50 (the amount of virus that will infect 50 percent of a target population) for that virus, preferably at least 10-fold less than the ID50 for that virus, more preferably at least 100-fold less than the ID50 for that virus, and still more preferably at least 1000-fold less than the ID50 for that virus.

The systems and methods disclosed may be better understood by reading the following description of non-limiting, exemplary embodiments with reference to the attached drawings, wherein like parts of each of the figures are identified by the same reference characters, and which are briefly described as follows.

### II. Systems

The systems disclosed herein are suitable for processing the amount of material (biologic product, such as a monoclonal antibody) produced by any suitable biological manufacturing process, including continuous or batch manufacturing.

In one embodiment, the system permits processing an amount of material (biologic product) produced by a system containing one or more integrated, continuous upstream operations including, for example, continuous (perfusion) cell culture, capture, viral inactivation, polishing or a combination thereof.

In certain embodiments, the systems and methods disclosed herein are suitable for use with iSKID (see, e.g., International Publication No. WO 2020/205559) in a small suite using a single-use flowpath. As referred to herein, the “iSKID” is a protein production platform that performs initial purification, viral inactivation, and polishing steps continuously over the course of a perfusion operation (for example, a 2-week high intensity perfusion operation). The systems and methods disclosed herein are not limited to applications with the iSKID but rather any system used to produce a biologic product, such as a monoclonal antibody.

In one embodiment, a VF-UFDF system (which may also be referred to as a VF-TFF system) is provided that includes both an initial purification assembly and a final purification assembly, wherein the assemblies are connected, coupled or otherwise integrated. In certain embodiments, the system is single-use.

The VF-UFDF system may further include a feed reservoir, e.g., a protein pool tank, which holds purified and polished protein, and in certain embodiments, is connected to the initial purification unit operation. The feed reservoir is well-mixed. The capacity of the protein pool tank may vary. In one embodiment, the protein pool tank has a capacity of between about 200 liters to about 5000 liters. In certain embodiments, the protein pool tank stores up to 40 kg of purified and polished monoclonal antibody, or purified and polished monoclonal antibody at a concentration between about 5 and about 20 g/L, or about 5 to about 15 g/L, or between about 8 about 12 g/L, or about 9 to about 11 g/L or about 10 g/L.

Optionally, the system may incorporate a pre-filtration step (e.g., microfiltration) to remove larger impurities or contaminants, such as protein aggregates.

In one embodiment, a system is provided that contains two skid bodies, including a first viral reduction filtration (VRF) skid in the initial purification suite and a second single-pass tangential flow filtration-diafiltration (SPTFF-DF) skid in the final purification suite.

In certain embodiments, the VRF skid includes a VRF pump, at least one VRF pre-filter and one or more VRF filters. In certain embodiments, the one or more VRF filters are placed in a VRF manifold. Applied pressure forces a portion of the fluid through the filter membrane and into the filtrate stream. In certain embodiments, the pump is replaced by a pressure fed vessel.

The VRF pump may vary. In one embodiment, the flow rate of the VRF pump is designed for 40 kilograms of product and more particularly, the flow rate is between about 80 liters/hour to 680 liters/hour, about 400 liters/hour to 560 liters/hour, or about 440 liters/hour to 520 liters/hour. Generally, the flow rate operating range on such a pump is in the range of about 5 liters/hour to 1200 liters/hour.

The one or more VRF membranes may vary. In operation, the product passes freely through the VRF membrane pores into the permeate, whereas viral particles, if present, are retained by the membrane.

The viral filter is capable of removing at least part of (such as at least 90%, 95%, 96%, 97%, 98%, or 99%, or 100%) viruses from a fluid (e.g., such as a liquid culture medium or fluid present in any of the processes described herein) including a recombinant antibody when the fluid is flowed through the filter.

Various filters are available for use in VRF, differing by mode of filtration, membrane area, membrane pore side, membrane material, module configuration and testing method.

Representative, non-limiting membrane materials include polymeric material such as, e.g., polyethylene, polypropylene, ethylene vinyl acetate copolymers, polytetrafluoroethylene, polycarbonate, poly vinyl chloride, polyesters, cellulose acetate, regenerated cellulose, cellulose composites, polysulphones, polyethersulfones, polyarylsulphones, polyphenylsulphones, polyacrylonitrile, polyvinylidene fluoride, non-woven and woven fabrics fibrous material, or of inorganic material.

Generally, the membrane area requirement is a function of how much (i.e., volume or mass) is intended to be filtered. In a particular embodiment, the filter is between about 1 and about 10 m2, or about 1 to about 8 m2, or about 8 m2, about 6 m2, about 4 m2 or about 2 m2 or less. In one embodiment, the filter is about 4 m2 or less for 15 kg or about 8 m2 or less for the 40 kg.

The membrane pore size may vary and in one embodiment, is between about 10 to about 100 nm, more particularly, about 15 to about 50 nm, even more particularly, about 20 to about 30 nm, even more particularly, about 20 nm.

In certain embodiments, the VRF membranes are pre-sterilized. In other embodiments, the VRF membranes are formed from materials that are suitable for sterilization. Examples of commercially available VRF membranes include Viresolve® Pro (Millipore), Planova 20N (Asahi Kasei) and Virosart (Satorius).

In one embodiment, the one or more VRF membranes are dead-end filters. In a dead-end filter, the flow of the liquid solution or suspension to be separated (or feed) is perpendicular to the membrane.

The capability of the VRF system may vary. Generally, viral reduction is measured by the ratio of the viral titer in the feed material to the relevant production fraction, referred to as log 10 reduction factor (LRF). In one embodiment, VRF permits a greater than about 6 LRF, greater than about 5 LRF, greater than about 4 LRF, greater than about 3 LRF or greater than about 2 LRF. The overall LRF of a single manufacturing process is based on the individual LRFs of each process step. In certain embodiments, no viral breakthrough is observed.

In certain embodiments, the VRF assembly is composed of a pre-filter attached to the VRF filter manifold and is linked via single-use aseptic connectors to a break-tank in final formulation.

Generally, the VRF operation is optimized to identify conditions that maximize volumetric throughput, minimize processing time and assure robust virus clearance.

In one embodiment, the volumetric throughput is between about 200 and about 1000 L/m2 and more particularly, between about 400 and about 600 L/m2. In certain embodiments, the volumetric throughput is between about 400 and about 450, about 450 and about 500, about 500 and about 550 or about 550 and about 600 L/m2.

In one embodiment, the mass throughout is between about 1 and about 10 kg/m2 and more particularly, between about 3 and about 5 kg.

In certain embodiments, the processing time is about 8 hours or less for up to 40 kg. For example, about 8 hours, about 7 hours, about 6 hours, about 5 hours, about 4 hours, about 3 hours, about 2 hours or about 1 hour or less.

In certain embodiments, the processing time is about 8 hours or less for about 15 kg. For example, about 8 hours, about 7 hours, about 6 hours, about 5 hours, about 4 hours, about 3 hours, about 2 hours or about 1 hour or less.

The SPTFF-DF assembly, which serves as the final purification component of the system, is composed of one or more SPTFF membranes, DF mixing tanks, DF membranes, sensors, and pumps. This second skid is supplied with concentrated buffers that are diluted inline with water to save space.

In one embodiment, the assembly includes a break tank, a diafiltration (DF) buffer concentrate, water for injection (WFI), a SPTFF-DF skid, UF/DF pool supply tubing and a UFDF pool tank.

According to this embodiment, the break tank is designed to provide a pressure break and safety in case flowrates are not perfectly matched between the viral filtration skid and the SPTFF-DF skid. In one embodiment, the break tank has a capacity of between about 20 liters and about 100 liters. The break tank is connected to both the VRF skid and the SPTFF-DF skid.

The diafiltration (DF) buffer concentrate and the WFI are designed to be mixed together so that the DF buffer concentrate is diluted to the appropriate concentration for buffer exchange.

The WFI is designed to provide water for flushing downstream equipment and for adjusting the concentration of the DF buffer concentrate to the appropriate concentration/strength that will be used in the process.

The SPTFF-DF skid is a skid unit that is pre-installed on a skid and is ready for easy transport having several aseptic connections for fluidly coupling the SPTFF-DF skid to other equipment and downstream of the viral filtration skid. In one embodiment, the SPTFF-DF skid includes a SPTFF pump, a SPTFF 1 membrane, a DF pool 1 tank, a DF pool 2 tank, a DF buffer pump, a WFI pump, an inline mixer, a DF pool 1 pump, a DF pool 2 pump, a DF membrane and an optional SPTFF 2 membrane. Additional equipment may be used without departing from the scope and spirit of the exemplary embodiment. Further, certain equipment may be combined, but the combined equipment may retain the same or similar functionalities without departing from the scope and spirit of the exemplary embodiment.

In one embodiment, after the first tank fills with concentrated product, the material starts diafiltering through a traditional TFF membrane while the SPTFF process continues filling the second tank. In one embodiment, the emptying and/or concentration of the first pool into the final UFDF pool need not be complete before the second pool begins diafiltering (i.e., mode 1). In another embodiment, when the first tank is finished (material diafiltered and emptied/concentrated into the final UFDF pool) and the second tank is filled, the second tank begins diafiltration (i.e., mode 2). Advantageously, this reduces the time required by almost half to complete the operation within a 12-hour window. In certain embodiments, the UF/DF time is reduced by about 50% compared to conventional operations, permitting the operation to process more material within the same time-frame. It also advantageously reduces the demands on pumps at any given time, allowing a smaller pumping system for the same total amount of material. Additionally, the use of a single-use flowpath reduces the time and resources needed for cleaning of the system after operations are complete.

The system can be operated in one of two modes, (i) a manual/partially automated mode or (ii) a fully automated mode. In both operational modes the VRF is run with the same automation with the only differences being the flow rate and membrane area.

Depending on loading volume and membrane capacity, more than one VRF membrane may be set up for the operation in the filter manifold and switched over to as needed.

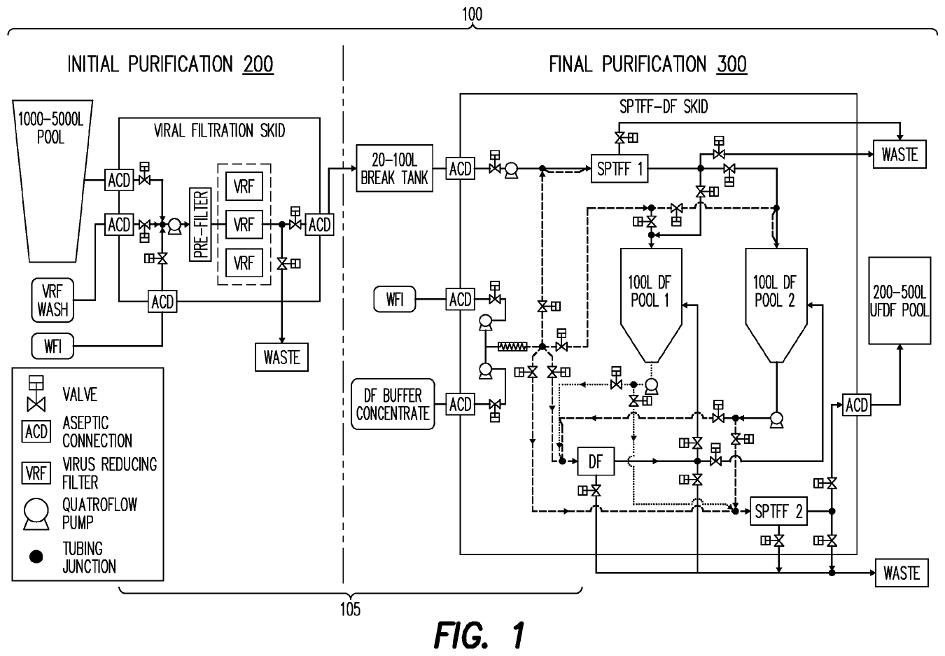

Referring now to FIGS. 1-3, the integrated single-use system 100, for performing an integrated viral filtration, concentration, and diafiltration process 105, includes the initial purification system 200 and the final purification system 300.

The initial purification system 200 includes a protein pool tank 210, a virus reduction filter (VRF) wash 220, a water for injection (WFI) tank 230, a viral filtration skid 240, and a break tank supply tubing 290. Although certain equipment has been included herein as being a part of the initial purification system 200, additional equipment may be used, or equipment may be combined, without departing from the scope and spirit of the exemplary embodiment.

The protein pool tank 210 is a tank that is designed to hold purified and polished protein and has a capacity of 500 liters to about 5000 liters according to some exemplary embodiments; however, the capacity of the protein pool tank 210 may be different in other embodiments. The protein pool tank 210 stores between about 5 kilogram to about 40 kilograms of purified and polished mAB and is a single-use mixer. The protein in the protein pool tank 210 is in the range of 5 grams/liter to about 15 grams/liter, and preferably about 10 grams/liter. The protein pool tank 210 is fluidly coupled to the viral filtration skid 240 via a protein pool tank discharge line 212, which connects to the viral filtration skid 240 at the protein pool tank aseptic connection 241.

The VRF wash 220 is designed to flush the integrated single-use system 100, and in particular, the viral filter 270 and viral filtration skid 240. The VRF wash 220 is fluidly coupled to the viral filtration skid 240 via a VRF wash discharge line 222, which connects to the viral filtration skid 240 at the VRF wash aseptic connection 244.

The WFI 230 is designed to provide water to flush the filters, as needed, for flushing the virus filter or pre-filter. The WFI 230 is fluidly coupled to the viral filtration skid 240 via a WFI discharge line 232, which connects to the viral filtration skid 240 at the WFI aseptic connection 247.

The viral filtration skid 240 is a skid unit that is pre-installed on a skid, is ready for easy transport, and has several aseptic connections for fluidly coupling the viral filtration skid 240 to other equipment. The viral filtration skid 240 includes a VRF pump 250, a VRF pre-filter 260, and one or more VRF membranes 270 that are optionally placed in a VRF manifold 271. Although certain equipment has been included herein as being a part of the viral filtration skid 240, additional equipment may be used, or equipment may be combined, without departing from the scope and spirit of the exemplary embodiment.

The VRF pump 250 is fluidly coupled to the protein pool tank aseptic connection 241 via a VRF pump suction line 242, which includes a VRF pump suction line control valve 243 located between the VRF pump 250 and the protein pool tank aseptic connection 241. The VRF pump 250 also is fluidly coupled to the VRF wash aseptic connection 244 via a VRF wash supply line 245 that extends from the VRF wash aseptic connection 244 to a VRF pump suction tubing junction 251, which is located between the VRF pump suction line control valve 243 and the VRF pump 250, and includes a VRF wash supply line control valve 246 positioned between the VRF pump suction tubing junction 251 and the VRF wash aseptic connection 244. Additionally, the VRF pump 250 is fluidly coupled to the WFI aseptic connection 247 via a WFI supply line 248 that extends from the WFI aseptic connection 247 to the VRF pump suction tubing junction 251, and includes a WFI supply line control valve 249 positioned between the VRF pump suction tubing junction 251 and the WFI aseptic connection 247. The VRF pump is a single use pump head integrated into the flow path. According to some exemplary embodiments, the VRF pump 250 is a QF1200SU Quattroflow low-shear pump with a feed flow between 80 liters/hour to 680 liters/hour (operating range between 10 liters/hour to 1200 liters/hour) or a Watson Marlow 600 pump having a feed flow of about 480 liters/hour (operating range between 5 liters/hour to 950 liters/hour); however, other embodiments may use other types of pumps.

The VRF pre-filter 260 is fluidly coupled to the VRF pump 250 via the VRF pump discharge line 254.

The VRF membrane 270 is fluidly coupled to the VRF pre-filter 260 via the VRF pre-filter discharge line 262. According to some exemplary embodiments, a plurality of VRF membranes 270A, 270B, 270C (or more) are fluidly coupled to the VRF pre-filter 260 in parallel to one another and optionally placed in a VRF manifold 271. The size of the VRF membrane 270 ranges from 1 meters-squared to 4 by 4 meters-squared (16 meters-squared). In certain embodiments, the VRF membrane 270 is the Planova 20N, the Planova BioEX, or the Viresolve Pro having a flux range between 20 LMH to 70 LMH, 35 LMH to 170 LMH, or 100 to 350 LMH, respectively. The VRF membrane 270 is fluidly coupled to a break tank aseptic connection 278 via the VRF membrane discharge line 276, which includes a VRF membrane discharge line control valve 277 positioned between the VRF membrane 270 and the break tank aseptic connection 278. A VRF waste discharge line 273 is coupled to the VRF membrane discharge line 276 at a VRF membrane discharge tubing junction 272, which is located between the VRF membrane discharge line control valve 277 and the VRF membrane 270, and includes a VRF waste discharge line control valve 274 positioned between the VRF membrane discharge tubing junction 272 and a waste 280.

The viral filtration skid 240 completes the initial purification system 200. The break tank aseptic connection 278 is fluidly coupled to the final purification system 300 via the break tank supply tubing 290. According to some exemplary embodiments, this break tank supply tubing 290 runs through a mousehole in a wall (not shown) which separates the initial purification suite 200 from the final purification suite 300.

The final purification system 300 includes a break tank 310, a diafiltration (DF) buffer concentrate 320, a water for injection (WFI) 330, a single-pass tangential flow filtration and diafiltration (SPTFF-DF) skid 340, a UFDF pool supply tubing 390, and a UFDF pool tank 395. Although certain equipment has been included herein as being a part of the final purification system 300, additional equipment may be used, or equipment may be combined, without departing from the scope and spirit of the exemplary embodiment.

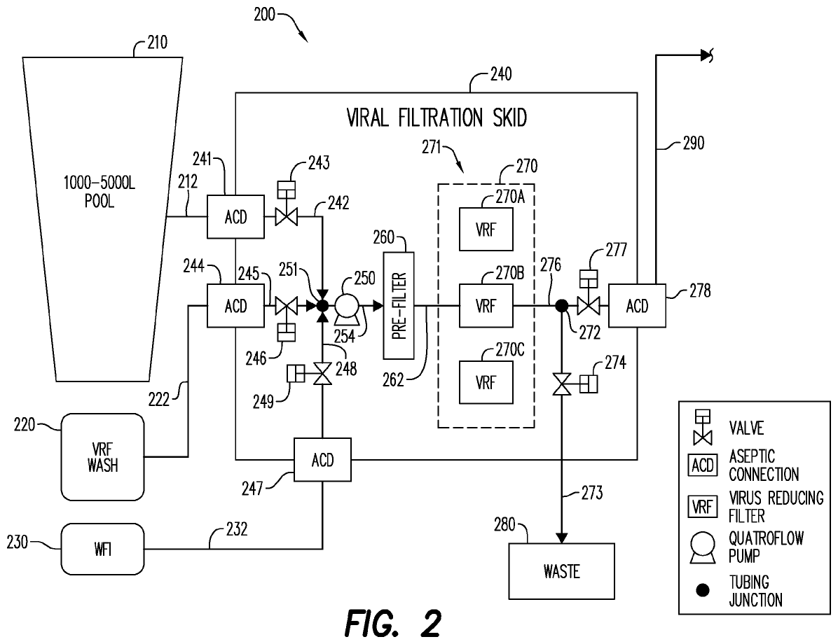

The break tank 310 is a tank that is designed to provide a pressure break and safety in case flowrates are not perfectly matched between the viral filtration skid 240 (FIG. 2) and the SPTFF-DF skid 340. The break tank has a capacity of 20 liters to about 100 liters according to some exemplary embodiments; however, the capacity of the break tank 310 may be different in other embodiments. The break tank 310 is fluidly coupled to the viral filtration skid 240 (FIG. 2) via the break tank supply tubing 290. The break tank 310 also is fluidly coupled to the SPTFF-DF skid 340 via a break tank discharge line 312, which connects to the SPTFF-DF skid 340 at the break tank aseptic connection 341.

The diafiltrate (DF) buffer concentrate 320 and the WFI 330 are designed to be mixed together so that the DF buffer concentrate 320 is diluted to the appropriate concentration. The DF buffer concentrate 320 is fluidly coupled to the SPTFF-DF skid 340 via a DF buffer concentrate discharge line 322, which connects to the SPTFF-DF skid 340 at the DF buffer concentrate aseptic connection 344.

The WFI 330 is designed to provide water for flushing downstream equipment and for adjusting the concentration of the DF buffer concentrate 320 to the appropriate concentration. The WFI 330 is fluidly coupled to the SPTFF-DF skid 340 via a WFI discharge line 332, which connects to the SPTFF-DF skid 340 at the WFI aseptic connection 347.

The SPTFF-DF skid 340 is a skid unit that is pre-installed on a skid and is ready for easy transport having several aseptic connections for fluidly coupling the SPTFF-DF skid 340 to other equipment downstream of the viral filtration skid 240. The SPTFF-DF skid 340 includes a SPTFF pump 3000, a SPTFF 1 membrane 3010, a DF pool 1 tank 3020, a DF pool 2 tank 3030, a DF buffer pump 3040, a WFI pump 3050, an inline mixer 3060, a DF pool 1 pump 3070, a DF pool 2 pump 3080, a DF membrane 3090, and an optional SPTFF 2 membrane 3100. Although certain equipment has been included herein as being a part of the SPTFF-DF skid 340, additional equipment may be used, or equipment may be combined, without departing from the scope and spirit of the exemplary embodiment.

The SPTFF pump 3000 is fluidly coupled to the break tank aseptic connection 341 via a SPTFF pump suction line 342, which includes a SPTFF pump suction line control valve 343 located between the SPTFF pump 3000 and the break tank aseptic connection 341. The SPTFF pump 3000 is a single use pump head integrated into the flow path. According to some exemplary embodiments, the SPTFF pump 3000 is a QF1200SU quattroflow low-shear pump with a feed flow range between 20 liters/hour to 1200 liters/hour.

The DF buffer pump 3040 is fluidly coupled to the DF buffer concentrate aseptic connection 344 via a DF buffer pump suction line 345, which includes a DF buffer pump suction line control valve 346 located between the DF buffer pump 3040 and the DF buffer concentrate aseptic connection 344. The DF buffer pump 3040 is a single use pump head integrated into the flow path. According to some exemplary embodiments, the DF buffer pump 3040 is a QF1200SU Quattroflow low-shear pump having an operating range between 20 liters/hour to 1200 liters/hour.

The WFI pump 3050 is fluidly coupled to the WFI aseptic connection 347 via a WFI pump suction line 348, which includes a WFI pump suction line control valve 349 located between the WFI pump 3050 and the WFI aseptic connection 347. The WFI pump 3050 is a single use pump head integrated into the flow path. According to some exemplary embodiments, the WFI pump 3050 is a QF1200SU Quattroflow low-shear pump having an operating range between 20 liters/hour to 1200 liters/hour.

The inline mixer 3060 is fluidly coupled to the DF buffer pump 3040 via a DF buffer pump discharge line 3042. The inline mixer 3060 also is fluidly coupled to the WFI pump 3050 via a WFI pump discharge line 3052 that extend from the WFI pump 3050 to a WFI pump discharge tubing junction 3041, which is located along the DF buffer pump discharge line 3042 between the inline mixer 3060 and the DF buffer pump 3040. The inline mixer 3060 is an in-line dilution system for the DF buffer concentrate 320 using the WFI 330 and includes helical in-line mixers.

The SPTFF 1 membrane 3010 is fluidly coupled to the SPTFF pump 3000 via a SPTFF pump discharge line 3002. The SPTFF 1 membrane 3010 also is fluidly coupled to the inline mixture 3060 via a SPTFF 1 membrane flush line 3062 that extends from the inline mixer 3060 to a SPTFF pump discharge tubing junction 3001, which is located along the SPTFF pump discharge line 3002 between the SPTFF pump 3000 and the SPTFF 1 membrane 3010, and includes a SPTFF 1 membrane flush control valve 3063. According to some exemplary embodiments, the SPTFF 1 unit 3010 is composed of a series of membranes that has a size capacity from about 0.9 meters-squared to about 20 meters-squared (e.g., Centrasette cassettes stacked in a Centrastak 100). The SPTFF 1 unit 3010 is set up in a 9-in-series configuration according to some exemplary embodiments. The size of the SPTFF 1 membrane 3010 is up to 20 meters-squared when designed for a 40 kilogram product through the integrated single-use system 100. The SPTFF 1 membrane 3010 is fluidly coupled to a waste 396 via a SPTFF permeate waste line 3011, which includes a SPTFF permeate waste control valve 3012 for controlling the flow of permeate from the SPTFF 1 membrane 3010 to the waste 396.

The DF pool 1 tank 3020 is fluidly coupled to the SPTFF 1 membrane 3010 via a retentate pool 1 line 3014, which includes a retentate pool 1 line control valve 3015 located between the SPTFF 1 membrane 3010 and the DF pool 1 tank 3020. The DF pool 1 tank 3020 is capable of operating in a first mode and a second mode, which are described in further detail in conjunction with the operation of the integrated single-use system 100. The DF pool 1 tank 3020 has a 20 to 100 liter tank capacity according to some exemplary embodiments. In certain embodiments, the connections of the SKIDS are asceptic. The DF pool 1 tank 3020 also is fluidly coupled to the inline mixer 3060 via a DF buffer pool 1 line 3064 that extends from the DF pool 1 tank 3020 to an inline mixer discharge tubing junction 3061, located between inline mixer 3060 and the SPTFF 1 membrane flush control valve 3063, and includes a DF pool tank control valve 3065 adjacent the inline mixer discharge tubing junction 3061 and a DF pool 1 tank control valve 3066 adjacent the DF pool 1 tank 3020.

The DF pool 2 tank 3030 also is fluidly coupled to the SPTFF 1 membrane 3010, via a retentate pool 2 line 3016 that extends from the DF pool 2 tank 3030 to a retentate pool 1 tubing junction 3013, located along the retentate pool 1 line 3014 between the SPTFF 1 membrane 3010 and the retentate pool 1 line control valve 3015, and includes a retentate pool 2 line control valve 3017 located between the retentate pool 1 tubing junction 3013 and the DF pool 2 tank 3030. The DF pool 2 tank 3030 is capable of operating in a first mode and a second mode, which are described in further detail in conjunction with the operation of the integrated single-use system 100. The DF pool 2 tank 3030 is similar to the DF pool 1 tank 3020 according to the exemplary embodiment. Additionally, a SPTFF 1 membrane retentate waste line 3018 extends from the retentate pool 1 tubing junction 3013 to the waste 396 and includes a SPTFF 1 membrane retentate waste line control valve 3019 for controlling the flow of retentate that goes to the waste 396. The DF pool 2 tank 3030 also is fluidly coupled to the inline mixer 3060 via a DF buffer pool 2 line 3067 that extends from a DF buffer pool 1 tubing junction 3068, located between the DF pool tank control valve 3065 and the DF pool 1 tank control valve 3066, to a DF buffer pool 2 tubing junction 3069, located between the DF pool 2 tank 3030 and the retentate pool 2 line control valve 3017, and includes a DF pool 2 tank control valve 3160.

The DF pool 1 pump 3070 is fluidly coupled to the DF pool 1 tank 3020 via a DF pool 1 pump suction line 3022. The DF pool 1 pump 3070 is a single use pump head integrated into the flow path. According to some exemplary embodiments, the DF pool 1 pump 3070 is a QF4400SU Quattroflow low-shear pump having an operating range between 150 liters/hour to 5000 liters/hour, a QF5050SU having an operating range between 50 liters/hour to 5000 liters/hour, or some other pump having the appropriate capacity depending upon the embodiment. The DF pool 2 pump 3080 is fluidly coupled to the DF pool 2 tank 3030 via a DF pool 2 pump suction line 3032. The DF pool 2 pump 3080 is a single use pump head integrated into the flow path. According to some exemplary embodiments, the DF pool 2 pump 3080 is the same or similar as the DF pool 1 pump 3070.

The DF membrane 3090 is fluidly coupled to the DF pool 1 pump 3070 via a DF pool 1 pump discharge line 3072 and includes a DF pool 1 pump control valve 3073. The DF membrane 3090 also is fluidly coupled to the DF pool 2 pump 3080 via a DF pool 2 pump discharge line 3082 that extends from the DF pool 2 pump 3080 to a DF membrane pool pump tubing junction 3074, located between the DF membrane 3090 and the DF pool 1 pump control valve 3073, and includes a DF pool 2 pump control valve 3083. The DF membrane 3090 also is fluidly coupled to the inline mixture 3060 via a DF membrane flush line 3161 that extends from the inline mixer discharge tubing junction 3061 to a DF membrane flush tubing junction 3162, which is located along the DF pool 1 pump discharge line 3072 between the DF membrane pump pool pump tubing junction 3074 and the DF membrane 3090, and includes a DF membrane flush control valve 3163. The DF membrane 3090 is capable of operating in a first mode and a second mode, which are described in further detail in conjunction with the operation of the integrated single-use system 100. According to some exemplary embodiments, the DF membrane 3090 is a Centrastak 100 membrane that has a size capacity of 0.9 meters-squared to 20 meters-squared. The DF membrane 3090 is fluidly coupled to a waste 397 via a DF permeate waste line 3091 and which includes a DF permeate waste control valve 3092 for controlling the flow of permeate from the DF membrane 3090 to the waste 397.

The DF membrane 3090 is fluidly coupled to the DF pool 1 tank 3020 via a DF pool 1 retentate recycle line 3093 extending from the DF membrane 3090 to the DF pool 1 tank 3020 and includes a DF pool 1 retentate recycle control valve 3094. The DF membrane 3090 also is fluidly coupled to the DF pool 2 tank 3030 via a DF pool 2 retentate recycle line 3095 extending from a DF pool tank retentate tubing junction 3096, located along the DF pool 1 retentate recycle line 3093 between the DF membrane 3090 and the DF pool 1 retentate recycle control valve 3094, to the DF pool 2 tank 3030, and includes a DF pool 2 retentate recycle control valve 3097. The retentate portion of the DF membrane 3090 is fluidly coupled to the waste 397 via a DF membrane retentate waste line 3098 extending from the DF pool tank retentate tubing junction 3096 to the waste 397, and includes a DF membrane retentate waste control valve 3099 for controlling the flow of retentate from the DF membrane 3090 to the waste 397. According to some embodiments, the DF membrane retentate waste line 3098 flows directly to the waste 397, or alternatively, may be combined with another waste line, such as the DF permeate waste line 3091.

The SPTFF 2 membrane 3100 is fluidly coupled to the DF pool 1 pump 3070 via a SPTFF 2 membrane pool 1 supply line 3076 that extends from the SPTFF 2 membrane 3100 to a SPTFF 2 membrane pool 1 supply tubing junction 3075, located along the DF pool 1 pump discharge line 3072 between the DF pool 1 pump 3070 and the DF pool 1 pump control valve 3073, and includes a SPTFF 2 membrane pool 1 supply line control valve 3077. The SPTFF 2 membrane 3100 also is fluidly coupled to the DF pool 2 pump 3080 via a SPTFF 2 membrane pool 2 supply line 3086 that extends from a SPTFF 2 membrane pool 2 supply tubing junction 3085, located along the DF pool 2 pump discharge line 3082 between the DF pool 2 pump 3080 and the DF pool 2 pump control valve 3083, to a secondary SPTFF 2 membrane pool 2 supply tubing junction 3087, located along the SPTFF 2 membrane pool 1 supply line 3076 between the SPTFF 2 membrane 3100 and the SPFF 2 membrane pool 1 supply line control valve 3077, and includes a SPTFF 2 membrane pool 2 supply line control valve 3088. The SPTFF 2 membrane 3100 also is fluidly coupled to the inline mixture 3060 via a SPTFF 2 membrane flush line 3164 that extends from the inline mixer discharge tubing junction 3061 to the SPTFF 2 membrane flush tubing junction 3087, and includes a SPTFF 2 membrane flush control valve 3165. The SPTFF 2 membrane 3100 is optional. According to some embodiments the SPTFF 2 membrane 3100 is similar to the SPTFF 1 membrane 3010. The SPTFF 2 membrane 3100 is fluidly coupled to the waste 397 via a SPTFF 2 permeate waste line 3101 and which includes a SPTFF 2 permeate waste control valve 3102 for controlling the flow of permeate from the SPTFF 2 membrane 3100 to the waste 397.

The SPTFF 2 membrane 3100 is fluidly coupled to a UFDF pool tank aseptic connection 380 via a SPTFF 2 retentate line 3105 extending from the SPTFF 2 membrane 3100 to the UFDF pool tank aseptic connection 380 and includes a SPTFF 2 retentate control valve 3106. The retentate portion of the SPTFF 2 membrane 3100 is fluidly coupled to the waste 397 via a SPTFF 2 retentate waste line 3107 extending from a SPTFF 2 membrane retentate tubing junction 3108, located along the SPTFF 2 retentate line 3105 between the SPTFF 2 membrane 3100 and the SPTFF 2 retentate control valve 3106, to the waste 397, and includes a SPTFF 2 membrane retentate waste control valve 3109. According to some embodiments, the SPTFF 2 retentate waste line 3107 flows directly to the waste 397, or alternatively, may be combined with another waste line, such as the DF permeate waste line 3091 or the SPTFF 2 permeate waste line 3101.

The UFDF pool tank 395 is fluidly coupled to the UFDF pool tank aseptic connection 380 via the UFDF pool supply tubing 390. The UFDF pool tank 395 is designed to have a capacity of 100 liters to 500 liters. The UFDF pool tank 395 receives the concentrated material from the SPTFF 2 membrane 3100, if included, or else from the DF pool 1 tank 3020 and the DF pool 2 tank 3030 after the DF membrane 3090 has recycled the retentate back to the DF pool 1 tank 3020 and the DF pool 2 tank 3030, respectively. After the UFDF pool tank 395 receives the concentrated material, the concentrated material will undergo final filtration and final formulation in accordance with known processes and procedures, not described herein.

Now that the schematics of FIGS. 1 through 3 have been described, the operation of the integrated single-use system 100 is now described. According to a brief overview of the integrated single-use system 100, the integrated single-use system 100 is designed to take between 5-40 kg of a single polished pool of monoclonal antibody (mAb) (i) through virus reduction filtration (VRF) that is performed by the viral filtration skid 200, and then (ii) through concentration by single-pass tangential flow filtration (SPTFF), buffer exchange by diafiltration (DF), an optional second concentration by SPTFF, to a pre-final filtration (UFDF) pool in a single batch in less than 12 hours (including non-operational setup and takedown time), which are all performed by the SPTFF-DF skid 300. This integrated single-use system 100 can be operated in one of two modes, a manual/partially automated mode that can process up to 15 kilograms in a single 12-hour batch, referred to as Mode 1 or first mode, or a fully automated mode that can process up to 40 kilograms in a single 12-hour batch, referred to a Mode 2 or second mode. All flowpaths, membranes, pump-heads, and connectors are made from sterile single-use materials.

The operations described below have estimated operating ranges for processing between 5 kilograms to 40 kilograms of antibody in a 12-hour time period; however, one skilled in the art may modify these operating ranges to process more or less than the 5 kilograms to 40 kilograms of antibody in a 12-hour time period or adjust the time period. Prior to commencing operation, the VRF membranes 270, the SPTFF 1 membrane 3010, the SPTFF 2 membrane 3100, if used, and the DF membrane 3090 along with associated lines and tanks are flushed and primed using a VRF wash 220 and/or WFI 230 and/or DF buffer and/or WFI 330.

In both operational modes, Mode 1 or Mode 2, the viral filtration skid 240 is run with the same automation with the only differences being the flow parameters and membrane area. Depending on loading volume and membrane capacity, more than one VRF membrane 270 may be set up for the operation in the VRF manifold 271, in parallel to one another, and switched over as needed.

In the initial purification 200, 5 kilograms to 40 kilograms of purified and polished mAb is stored in the protein pool tank 210, which is a 200-5000 L single-use mixer (SUM) or storage tank at about 10 g/L (7-13 g/L) according to an exemplary embodiment. The VRF pump 250 pumps the material from the protein pool tank to the VRF pre-filter 260, then to the VRF membrane 270, at a feed flow of 80-680 liters/hour. The VRF pump 250 is a QF1200SU Quattroflow low-shear pump with a single use pump head integrated into the flow path having an operational range between 20 liters/hour to 1200 liters/hour. 0.9 meters-squared to 8 meters-squared of VRF membrane 270 is used and loaded up to a capacity of 385 liters/meters-squared, which can be up to 600 liters/meters-squared, with a target flux of 64 LMH, which can be up to 300 LMH. The flowthrough continues from the VRF membrane 270 to the break tank 310, which is a 20 liter to 100 liter tank that is part of the final purification 300. The initial purification 200 runs continuously for 6 hours or until all the starting material that is stored within the protein pool tank 210 is processed and starts filling up the break tank 310. During operation of the initial purification 200, the VRF pump suction line control valve 243 and the VRF membrane discharge line control valve 277 are in the open position to allow flow therethrough, while the VRF wash supply line control valve 246, the WFI supply line control valve 249, and the VRF waste discharge line control valve 274 are in the close position to prevent flow therethrough.

In certain embodiments, after all feed material has been loaded, the VRF membrane 270 is flushed with 10 liters/meters-squared VRF wash 220. Before and after the operation of the initial purification 200 when a wash is performed on the initial purification 200, the VRF pump suction line control valve 243 and the WFI supply line control valve 249 are placed in the close position to prevent flow therethrough, while the VRF wash supply line control valve 246, and the VRF membrane discharge line control valve 277 are placed in the open position to allow flow therethrough. The wash fluid exits the initial purification 200 to break tank 310.

As the break tank 310 begins filling, the SPTFF pump 3000 on the SPTFF-DF skid 340 begins pumping material from the break tank 310 through the SPTFF 1 membrane 3010 at feed flow equivalent to the VRF pump 250, which is between 80 liters/hour to 680 liters/hour.

Operation of the SPTFF-DF skid 340 changes from this step forward based on whether operations are in mode 1 or in mode 2, in which operations are quite different. When the SPTFF-DF skid 340 is operated in the less automated mode 1, the SPTFF-DF skid 340 can process up to 15 kilograms of protein and splits the material that was in the protein pool tank 210 into two sequential DF pool tanks 3020, 3030, which are the DF pool 1 tank 3020 and the DF pool 2 tank 3030. When processing more than 15 kilograms of protein, the SPTFF-DF skid 340 operates in mode 2, where the SPTFF-DF skid 340 performs many shorter diafiltration (DF) steps by switching back and forth between processing material in DF pool 1 tank 3020 and DF pool 2 tank 3030.

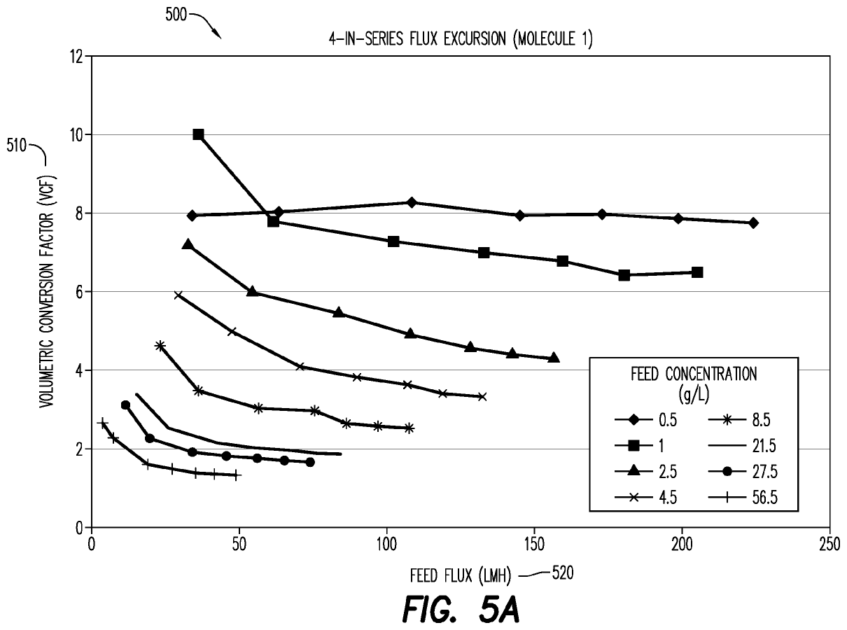

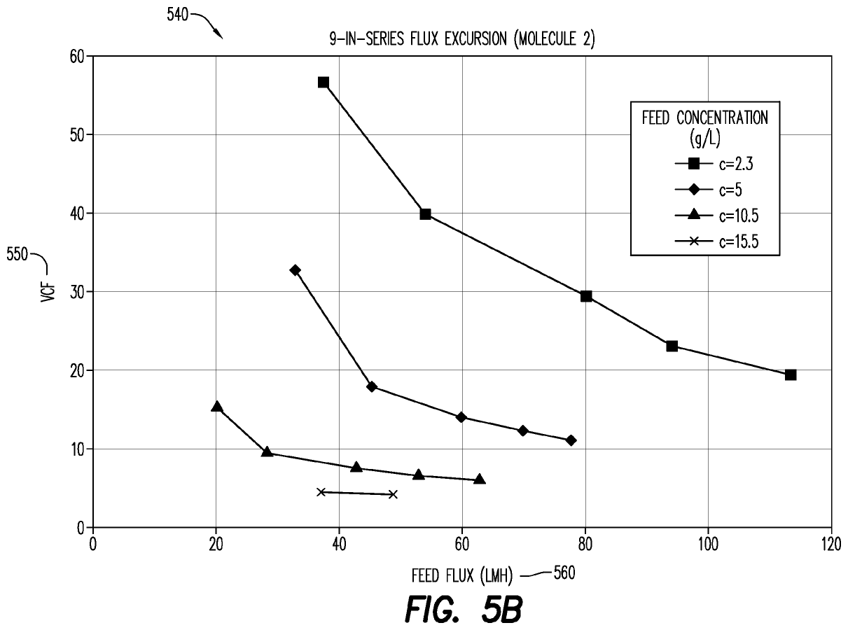

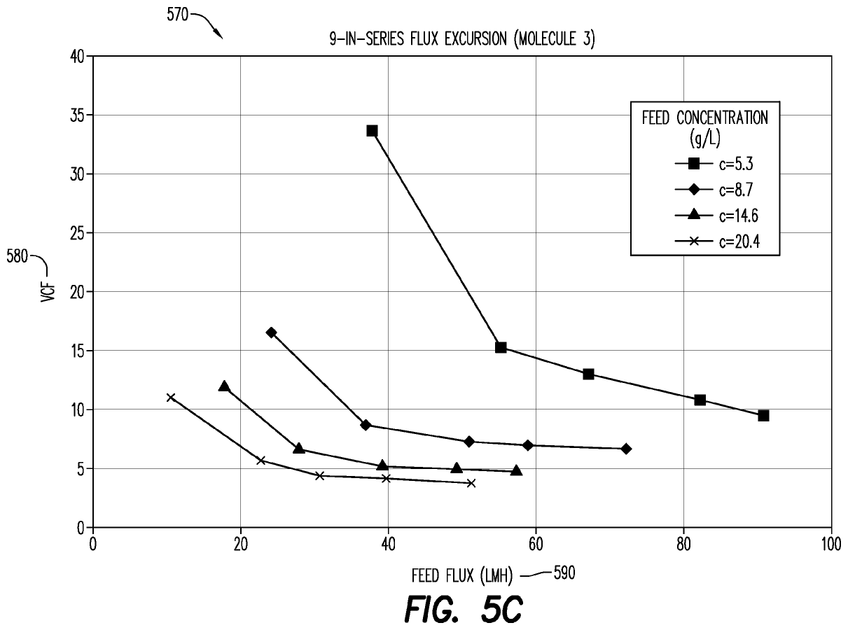

Operation of the SPTFF-DF skid 340 in mode 1 is now described. The SPTFF 1 membrane 3010 is set up in a 9-in-series configuration. In other embodiments, the series could be a 4-in-series, 5-in-series, 6-in-series, 7-in-series, 8-in-series or 9-in series. The operation of the SPTFF-DF skid 340 uses a QF1200SU Quattroflow low-shear pump with a single use pump head integrated into the flow path and has an operational range between 20 liters/hour to 1200 liters/hour. When processing 15 kilograms of protein, the SPTFF 1 membrane 3010 has approximately 9 meters-squared membrane area, which operationally ranges between 3 meters-squared and 20 meters-squared. A constant flux, which can be between 10 LMH and 50 LMH, is targeted in order to achieve a consistent volumetric concentration factor (variable), if the permeate flux decreases over the course of the operation the feed flux can be modified to maintain a constant VCF. For example, with Molecule 1, using a 4-in-series membrane configuration, at a starting concentration of 10 g/L a feed flux of 25.5 LMH was targeted to achieve an 8×VCF and target concentration of 80 grams/liter. The target concentration, flux and membrane area requirements should be determined prior to operation in development via flux excursion experiments as shown for Molecules 1, 2, and 3 in FIGS. 5A, 5B, and 5C respectively. The membrane area for SPTFF 1 membrane 3010 may be increased or decreased depending on molecule specific properties in order to maintain a relatively constant flux consistent with VRF flow rates. The membrane holder is the Centrastak 100, which can accommodate a membrane area of 0.9 meters squared to 20 meters-squared. The concentrated material, or the retentate of SPTFF 1 membrane 3010, flows out the SPTFF 1 membrane 3010 to the DF pool 1 tank 3020, which is an up to 100 liter single-use mixing tank, that will be used for diafiltration. During this time, the retentate pool 1 line control valve 3015 is in the open position, while the retentate pool 2 line control valve 3017 and the SPTFF 1 membrane retentate waste line control valve 3019 are in the close position. Halfway through processing the material from the initial purification 200 within the SPTFF 1 membrane 3010, the retentate pool 1 line control valve 3015 switches to the close position and the retentate pool 2 line control valve 3017 switches to the open position to stop loading the DF pool 1 tank 3020 and start loading the DF pool 2 tank 3030. The DF pool 2 tank 3030 is a single-use mixing tank that is similar or same in size to the DF pool 1 tank 3020 according to some exemplary embodiments. Once the break tank 310 empties, the SPTFF 1 membrane 3010 is flushed with 2× the membrane hold-up volume of wash buffer, which is a mixture of the DF buffer concentrate 320 and the WFI wash 330.

Halfway through the operation of the SPTFF 1 membrane 3010, once the retentate pool 1 line control valve 3015 switches to the close position and the retentate pool 2 line control valve 3017 switches to the open position, the DF pool 1 tank 3020 starts performing diafiltration. The DF pool 1 pump 3070 starts pumping material from the DF pool 1 tank 3020 and passes the material through the DF membrane 3090, which has an area between 2 meters-squared and 20 meters-squared according to some exemplary embodiments, at a target feed flow of 800 liters/hour to 7000 liters/hour or 360 LMH. The operation of the DF membrane 3090 requires the DF pool 1 pump 3070 to be a larger sized pump than the SPTFF feed pump. According to some exemplary embodiments, the DF pool 1 pump is a QF4400SU quattroflow low-shear pump that is a single use pump head integrated into the flow path and has an operational range of 150 liters/hour to 5000 liters/hour. As with the area of the SPTFF 1 membrane 3010, the area of the DF membrane 3090 can be adjusted on a molecule specific basis. The membrane holder is a Centrastak 100, which can accommodate 0.90 meters-squared to 20 meters-squared. At 80 grams/liter flow, diafiltration is expected to have an average 10% conversion, or 36 LMH flux. The diafiltration buffer concentrate 320 is mixed in-line with WFI 330 and the resulting mixture is added to the DF pool 1 tank 3020 at a rate automatically matching the permeate flow rate exiting the DF membrane 3090 through the DF permeate waste line 3091. After 7 to 10 diavolumes (DVs) of the mixture being added to the DF pool 1 tank 3020, the material should be appropriately buffer exchanged, which can be detected via one or more in-tank/in-line sensors (not shown). For 8 DVs of mixture being added, this process of using the DF pool 1 tank 3020 and the DF membrane 3090 is expected to take approximately 3 hours. The retentate of the DF membrane 3090 recycles back to the DF pool 1 tank 3020 through the DF pool 1 retentate recycle line 3093. Upon completion of adding the mixture of DF buffer concentrate 320 and WFI 330 to the DF Pool 1 tank 3020 and appropriately completing the buffer exchange, a similar process is repeated with the DF pool 2 tank 3030 after the SPTFF 1 membrane 3010 has finished with the second half of the operation.

An optional SPTFF 2 membrane 3100 can be used to achieve the final target concentration (the final desired concentration will dictate the required path length/membrane area for this concentration step). The material residing in the DF pool 1 tank 3020 after buffering is pumped through the DF Pool 1 pump 3070 to the SPTFF 2 membrane 3100 via the SPTFF 2 membrane pool 1 supply line 3076. Upon the material flowing through the SPTFF 2 membrane 3100, the retentate concentrated material flows to the UFDF pool tank 395, which is a 100 liter to 500 liter tank, prior to final filtration and final formulation. Once the material from the DF Pool 1 tank 3020 is processed through the SPTFF 2 membrane 3090, a similar process through the SPTFF 2 membrane 3100 is repeated with the material residing within DF pool 2 tank 3030. In an alternative embodiment where the SPTFF 2 membrane 3100 is not present, the buffered material within the DF pool 1 tank 3020 and the DF pool 2 tank 3030 are sequentially transferred from the respective tank 3020, 3030 to the UFDF pool tank 395.

Alternatively, the SPTFF-DF skid 340 can operate in mode 2, which is now described below. The general operational principles of the SPTFF-DF skid 340 operating in mode 2 are identical to when the SPTFF-DF skid 340 is operating in mode 1; however, instead of filling the DF pool 1 tank 3020 completely and switching halfway through the material being sent from the protein pool tank 210 to fill the DF pool 2 tank 3030, the filling of the DF pool 1 tank 3020 and the DF pool 2 tank 3030 are switched many times throughout the operation of the SPTFF-DF skid 340 in mode 2 so then many less filled DF pool tanks 3020, 3030 are processed back and forth repeatedly. While one tank of the DF pool 1 tank 3020 and the DF pool 2 tank 3030 is performing diafiltration through the DF membrane 3090 DF and then sending material to the SPTFF 2 membrane 3100 (or directly to the UFDF pool tank 395), the other tank is filling. The more frequently switched, the more material that can be processed in the same period of time. This mode 2 would require additional automation for the valves to switch fluid flow between the DF pool 1 tank 3020 and the DF pool 2 tank 3030 (valve switching) while monitoring and reacting to volume totals, pH, conductivity, concentration, and flow rates.

Once mode 1 or mode 2 operations are completed depending upon the mode selected to operate the SPTFF-DF skid 340, the integrated single-use system 100 is then flushed and the single-use flow path is discarded.

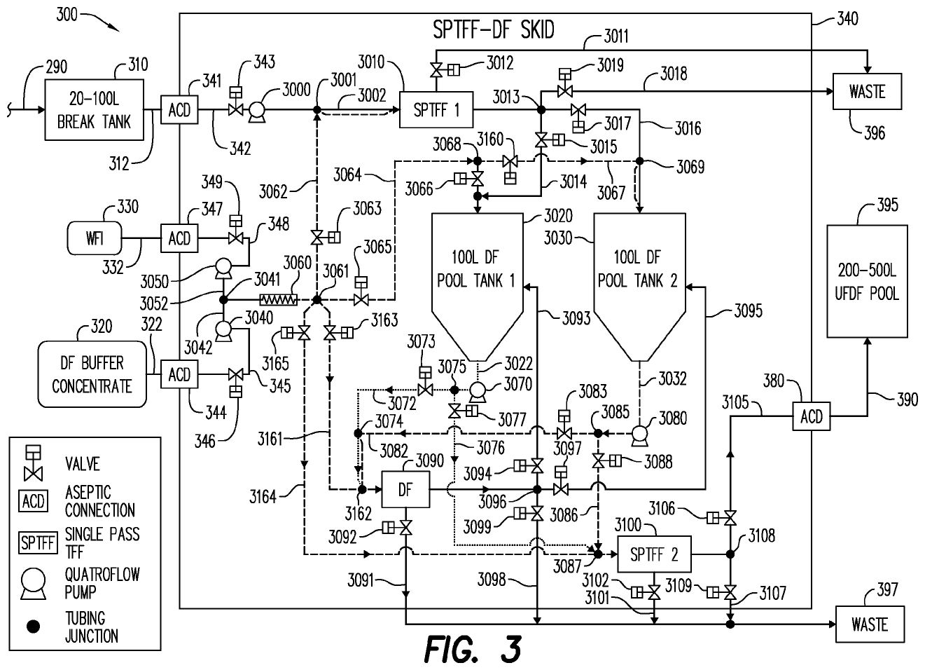

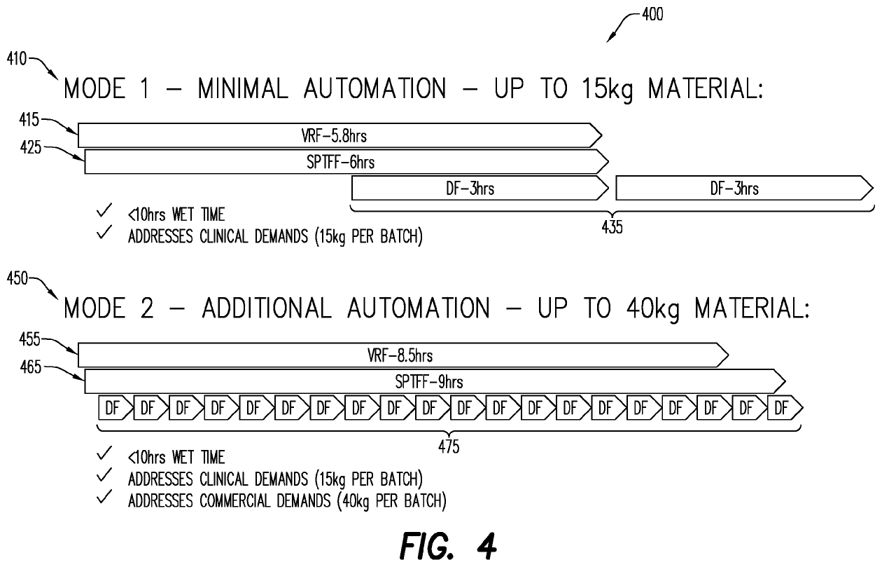

FIG. 4 shows a mode 1 and 2 pictorial time representation 400 comparing the operating times of the integrated single-use system when operating in mode 1 410 or mode 2 450 in accordance with an exemplary embodiment. Referring to FIG. 4, mode 1 410 operates with minimal automation and processes up to 15 kilograms of material, while mode 2 operates with additional automation and processes up to 40 kilograms of material. Mode 1 410 includes operation of the virus reduction filtration (VRF) 415 in the viral filtration skid 240, the single-pass tangential flow filtration (SPTFF) 425 in a portion of the SPTFF-DF skid 340, and diafiltration (DF) 435 in a portion of the SPTFF-DF skid 340. The VRF 415, the SPTFF 425 and the DF 435 collectively form the entire process of the integrated single-use system 100 (FIG. 1). Accordingly, the VRF 415 starts the process and takes 5.8 hours to complete in mode 1 410, which is for up to 15 kilograms of material. The SPTFF 425 of mode 1 410 commences shortly after the start of the VRF 415 and takes 6 hours to complete. The DF 435 starts at the halfway point of the SPTFF 415 and takes 6 hours to complete, 3 hours for DF pool 1 tank 3020 (FIG. 3) and 3 hours for DF pool 2 tank 3030 (FIG. 3). Hence the entire process of the integrated single-use system 100 (FIG. 1) operating in mode 1 410 takes less than 10 hours to complete for processing up to 15 kilograms of material, which is a sufficient quantity for addressing clinical demands.

Mode 2 450 includes operation of the virus reduction filtration (VRF) 455 in the viral filtration skid 240, the single-pass tangential flow filtration (SPTFF) 465 in a portion of the SPTFF-DF skid 340, and diafiltration (DF) 475 in a portion of the SPTFF-DF skid 340. The VRF 455, the SPTFF 465 and the DF 475 collectively form the entire process of the integrated single-use system 100 (FIG. 1). Accordingly, the VRF 455 starts the process and takes 8.5 hours to complete in mode 2 450, which is for up to 40 kilograms of material. The VRF 455 of mode 2 450 takes more time than the VRF 415 of mode 1 410 due to the increase amount of material that is to be processed in the same manner. The SPTFF 465 of mode 2 450 commences shortly after the start of the VRF 455 and takes 9 hours to complete. The DF 475 starts shortly after the start of the SPTFF 465 and takes about 9 hours to complete, 10 cycles of each DF pool 1 tank 3020 (FIG. 3) and DF pool 2 tank 3030 (FIG. 3), where each cycle is a little less than 55 minutes. Hence the entire process of the integrated single-use system 100 (FIG. 1) operating in mode 2 450 takes less than 10 hours to complete for processing up to 40 kilograms of material, which is a sufficient quantity for addressing clinical demands and for addressing commercial demands. Mode 2 450 is capable of processing much more material than mode 1 410 because the DF 475 is able to start shortly after the start of the SPTFF 465 and operates in short iterative cycles between DF pool 1 tank 3020 (FIG. 3) and DF pool 2 tank 3030 (FIG. 3).

FIG. 5A shows a graph 500 of a Volumetric Conversion Factor (VCF) Pall 4-In-Series Flux Excursion (Molecule 1), which illustrates the volumetric conversion factor (VCF) 510 versus feed flux 520 for Molecule 1 at various starting concentrations using a Pall 4-in-series membrane for the single-pass tangential flow filtration (SPTFF) 1 membrane 3010 (FIG. 3) in accordance with an exemplary embodiment. Essentially what can be seen from this graph 500 is that at high starting concentrations the maximum potential conversion factor decreases but also that a lower flux has a greater influence. If starting with a high concentration material, the lower the flux the more concentration you get. If starting at a lower concentration material, a lower flux has less of an impact on the conversion factor. These experiments can be conducted prior to performing large scale concentration of a product in order to determine the optimal flux for a given molecule for a desired conversion factor and final concentration. Thus, at the starting concentration of the protein pool tank 210 (˜10 grams/liter) the flux would have to be quite low (˜10 LMH) in order to concentrate to the desired 80 grams/liter. In order to run the integrated single-use system 100 (FIG. 1), or VFTFF system, at the desired flux, a 9-in-series (or in certain embodiments, less than a 9-in-series, e.g., a 4-in series or greater but less than 9) or more than a 9-in-series) SPTFF 1 membrane 3010 (FIG. 3) configuration would be needed.

FIG. 5B shows a graph 540 of a Volumetric Conversion Factor (VCF) Pall 9-In-Series (Molecule 2) Flux Excursion, which illustrates the volumetric conversion factor (VCF) 550 versus feed flux 560 for Molecule 2 at various starting concentrations using a Pall 9-in-series membrane for the SPTFF 1 membrane (FIG. 3) in accordance with an exemplary embodiment. Referring to FIG. 5B, it can be seen that at high starting concentrations the maximum potential conversion factor decreases but also that a lower flux has a greater influence. The 9-in-series SPTFF is able to achieve higher concentrations at similar fluxes to the 4-in-series allowing it to more easily pair with a slower VF system, making it an ideal candidate for the SPTFF arrangement in the VF-TFF system. One limitation is that at higher feed concentrations the maximum feed flux for the system is lower due to pressure thresholds. If this cannot be accommodated for with additional membrane area, then a smaller (7-in-series or 4-in-series, for example) system could be used instead, especially if less concentration is required for the DF step. This set of flux excursions demonstrates that a feed flux in the operating range for the integrated single-use system 100 (FIG. 1), or VFTFF system, could achieve the desired concentration factor at a reasonable feed flux.

FIG. 5C shows a graph 570 of a Volumetric Conversion Factor (VCF) Pall 9-In-Series (Molecule 3) Flux Excursion, which illustrates the volumetric conversion factor (VCF) 580 versus feed flux 590 for Molecule 3 at various starting concentrations using a Pall 9-in-series membrane for the SPTFF 1 membrane (FIG. 3) in accordance with an exemplary embodiment. Referring to FIG. 5C, it can be seen that at high starting concentrations the maximum potential conversion factor decreases but also that a lower flux has a greater influence. The 9-in-series SPTFF is able to achieve higher concentrations at similar fluxes to the 4-in-series allowing it to more easily pair with a slower VF system, making it an ideal candidate for the SPTFF arrangement in the VF-TFF system. If a higher VF flow is used or there is a higher feed concentration then a smaller (7-in-series or 4-in-series, for example) system could be used instead, especially if less concentration is required for the DF step. This set of flux excursions demonstrates that a feed flux in the operating range for the integrated single-use system 100 (FIG. 1), or VFTFF system, could achieve the desired concentration factor at a reasonable feed flux. In view of the foregoing, the integrated single-use system 100 provides for at least one of de-risking, space-savings, and time-savings when processing a biologic product (e.g., a protein) through filtration processes.

With respect to de-risking, one of the principles of this system is that it can process all the material in a single batch. By performing viral filtration all at once, as opposed to continuously or periodically, the risk of viral breakthrough is minimized, as are operational issues due to changes in flow or pressure. This also avoids regulatory concerns over batch definition, as there are no “sub-batches.”

With respect to space-savings, performing SPTFF and VRF in tandem with matching flow rates reduces the space required to hold the virus-filtered material, thereby requiring only a small break tank that has a capacity between 20 liters to 100 liters. The size of the tanks required for diafiltration (DF) is also minimized by targeting a high initial concentration factor, that is 8-fold in some exemplary embodiments, bringing the VRF pool from 10 grams/liter to 80 grams/liter. In a particular embodiment, the tank(s) is/are about 350 L. Space savings also are achieved by using an in-line dilution system for the DF buffer, via helical in-line mixers according to some exemplary embodiments.

In a particular embodiment, the system is run in mode 1 and the size of the DF tank is at least about 6-times smaller than the starting tank. For example, about 350 L×2 tanks compared to a 2000 L starting tank.

In a particular embodiment, the system is run in mode 2 and the size of the tanks required for DF is at least about 8, at least about 10, at least about 12, at least about 14, least about 16, or at least about 20-times smaller than the starting tank.

With respect to time-savings, by running VRF in tandem with the SPTFF there is an automatic time-savings of at least a day as these processes are traditionally run on separate days, with VRF run on the first day and UFDF run the next day. In addition, time-savings is also achieved by using a two-tank DF system. After the first DF tank fills with concentrated product, the material starts diafiltering through a traditional TFF membrane while the SPTFF process continues filling the second DF tank. When the first DF tank is finished (material diafiltered and emptied) and the second tank is filled, the second DF tank begins diafiltration. This process can cut the UF/DF time by almost half when operating in Mode 2 in order to complete the operations within the 12-hour window (FIG. 4).

Another benefit is that the system reduces the demands on pumps at any given time, allowing for a smaller pumping system, thereby reducing costs. Additionally, the use of a single-use flow path reduces the time and resources needed for cleaning of the system after operations are complete.

### III. Methods

Also disclosed herein is a method for removing viruses by filtration and concentrating/buffer exchanging a biologic product (e.g., a protein) in an integrated, continuous fashion.

In one embodiment, the method comprises (i) providing a biologic product in solution; and (ii) subjecting the solution to (a) virus reduction filtration (VRF), (b) concentration by single-pass tangential flow filtration (SPTFF), (c) buffer exchange by diafiltration (DF), and (d) optionally, second concentration by SPTFF.

As noted above, filtration of viruses that may be present in a composition including a biologic product that is intended for use in a biopharmaceutical product is an important aspect of quality control. The biologic product can be, for example, a protein, a nucleic acid, a carbohydrate, a lipid, or a biomaterial, among other substances. The protein can be, for example, a therapeutic protein, such as an antibody, an antibody fragment, an antibody derivative, a cytokine, a growth factor, a hormone, an enzyme, or a blood coagulation factor, among others, or a vaccine protein, such as an antigenic protein, among others. The biologic product can be produced by a living system, such as a cell, tissue, or organism, e.g. by a mammalian cell, a plant cell, or a bacterial cell, among others. The biologic product can be produced by a homogeneous process, e.g. suspension culture based on use of a stirred-tank bioreactor, air-lift bioreactor, or wave bioreactor, or a heterogeneous process, e.g. adherent culture based on a microcarrier-based system, a packed bed bioreactor, or a hollow-fiber bioreactor, as carried out in a discontinuous mode, e.g. batch cultivation or fed-batch cultivation, or in a continuous mode, e.g. continuous cultivation with perfusion, and as carried out at any suitable scale, e.g. laboratory, pilot, or production scale. The virus may be one that can infect bacteria (i.e. a “bacteriophage,” also termed a “phage”), or a human and/or an animal, e.g. the individual human or animal for which the biologic product is intended for administration, among others. The virus may have been introduced into the composition including the biologic product from an exogenous source, e.g. by inadvertent failure to maintain sterility, or from an endogenous source, e.g. the living system used to make the biologic product.

The method can be used to ensure that a virus that may have been present during the manufacture of the biologic product, for example based on viral contamination, is removed or eliminated. To the extent that multiple different types of viruses and/or multiple active particles of a given type of virus may be present, the method can be used to remove the multiple different types and/or multiple active particles of a given type. Thus, for example, the method can be used to ensure that a biopharmaceutical product that ultimately includes the biologic product will not include active particles of virus of any type in any amount above an acceptable limit, e.g. that the biopharmaceutical product will be free of active particles of virus.

In certain embodiments, the method involves viral filtration, concentration, and diafiltration in a single batch in less than 12 hours (including non-operational setup and takedown time). In one embodiment, the method takes less than 12 hours, less than 11 hours, less than 10 hours, less than 9 hours, less than 8 hours, less than 7 hours, less than 6 hour or 5 hours or less.

In one embodiment, (i) providing a biologic product in solution involves providing between about 5 to about 40 kg of purified and polished monoclonal antibody (mAb) which is stored in a 500-2000 L single-use mixer (SUM) or storage tank at about 10 g/L (5-15 g/L).

In one embodiment, (ii) subjecting the solution to (a) virus reduction filtration (VRF), involves the material passing through a pre-filter to a VRF membrane (Planova BioEX) at a feed flow of between about 80 and about 680 L/hr using a QF1200SU Quattroflow low-shear pump with a single use pump head integrated into the flow path (operational range 20-1200 L/hr). 0.9-8 m2 of VRF membrane is used and loaded up to a capacity of 385 L/m2 (up to 600 L/m2), with a target flux of 64 LMH (up to 150 LMH). This process runs continuously for 6 hours or until all the starting material is processed. After all feed material has been loaded the membrane is flushed with 10 L/m2 wash buffer.

The flowthrough continues through to a 20-100 L break tank in final purification. As the break tank begins filling the SPTFF pump on the second skid begins pumping material through the SPTFF membrane at feed flow equivalent to the VRF feed pump, 80-680 L/hr.

The method of (b) concentration by single-pass tangential flow filtration (SPTFF) may vary. That is, the SPTFF-DF operates differently depending on the operational mode. When operated in the less automated Mode 1, it can process up to 15 kg and splits the VRF pool into two sequential DF pools. When processing more than 15 kg, in Mode 2, it performs many shorter DF steps, switching back and forth between DF Pool 1 and DF Pool 2.

Mode 1: The SPTFF membrane is set up in a 4- to 9-in-series configuration. The SPTFF operation uses a QF1200SU quattroflow low-shear pump with a single use pump head integrated into the flow path (operational range 20-1200 L/hr). Target concentration and flux should be determined prior to operation in development via a flux excursion experiment similar to the one in FIG. 1. The membrane area may be increased or decreased depending on molecule specific properties in order to maintain a relatively constant flux. The membrane holder is the Centrastak 100, which can accommodate 0.9-20 m2. The concentrated material flows out the SPTFF to the first of two 100 L single-use mixing tanks for diafiltration. Halfway through processing the material from VRF a valve at the outlet of the SPTFF switches to start loading the second 100 L single-use mixing tank. After the break tank empties the membrane is flushed with 2× the membrane hold-up volume of wash buffer.

Halfway through the SPTFF operation, once the outlet valve of SPTFF switches to the second 100 L tank, the first tank starts performing diafiltration. A pump starts passing the material over 0.9-20 m2 of TFF membrane a target feed flow of 800-7000 L/hr or 360 LMH. The DF operation requires a larger QF4400SU quattroflow low-shear pump a single use pump head integrated into the flow path (operational range 150-5000 L/hr). As with the SPTFF operation the DF operation membrane area can be adjusted on a molecule specific basis. The membrane holder is a Centrastak 100, which can accommodate 0.9-20 m2. At 80 g/L diafiltration is expected to have an average 10% conversion (36 LMH flux). The diafiltration buffer concentrate is mixed in-line with water and added to the mixing tank at a rate automatically matching the permeate flow rate. After 5-10 diavolumes (DVs) the material should be appropriately buffer exchanged (detect via in-tank/in-line sensors). For 8 DVs this process is expected to take approximately 3 hours. The process is repeated with the second diafiltration tank after the SPTFF process has finished.

An optional second SPTFF membrane can be used for UF2 to achieve the final target concentration (the final desired concentration will dictate the required path length for this concentration step). This concentrated material flows through to a final 200-500 L pool prior to final filtration and final formulation.

Mode 2: The general operational principles are identical but instead of filling the DF Pool completely and switching halfway through the VRF Pool the DF Pools are switched many times throughout the operation for many smaller DF pools. While one tank is performing DF and then sending material to the second SPTFF (or directly to the final pool), the other tank is filling. The more frequently switched, the more material that can be processed in the same period of time.

The system is then flushed, and the single-use flow path is discarded. The systems and methods disclosed herein may be used to provide an aqueous formulation containing any biologic product of interest (e.g., a protein).

The method offers the advantages discussed above with respect to the system, i.e., de-risking, space-savings, and time-savings when processing a biologic product (e.g., a protein) through filtration processes.

Methods of manufacturing or producing of the biologic product of interest known in the art may be used in combination with the systems and methods of filtering a fluid feed described herein. For example, a person of skill in the art knows how to manufacture or produce biologic products, such as recombinant proteins, using fermentation. In certain embodiments, the production of biologic product of interest comprises cultivating the eukaryotic cell expressing the biologic product of interest in cell culture. Cultivating the eukaryotic cell expressing the biologic product of interest in cell culture may comprise maintaining the eukaryotic cells in a suitable medium and under conditions that allow growth and/or protein production/expression. The biologic product of interest may be produced by fed-batch or continuous cell culture. Thus, the eukaryotic cells may be cultivated in a fed-batch or continuous cell culture, preferably in a continuous cell culture.

In certain embodiments, the eukaryotic host cells are yeast cells. In one embodiment, the eukaryotic host cell is a mammalian cell. Mammalian cells as used herein are mammalian cells lines suitable for the production of a secreted recombinant therapeutic protein and may hence also be referred to as “host cells”. In certain embodiments, the mammalian cells are rodent cells such as hamster cells. The mammalian cells are isolated cells or cell lines. In certain embodiments, the mammalian cells are transformed and/or immortalized cell lines. In certain embodiments, the mammalian cells are adapted to serial passages in cell culture and do not include primary non-transformed cells or cells that are part of an organ structure. In certain embodiments, the mammalian cells are BHK21, BHK TK-, Jurkat cells, 293 cells, HeLa cells, CV-1 cells, 3T3 cells, CHO, CHO-K1, CHO-DXB11 (also referred to as CHO-DUKX or DuxB11), a CHO-S cell and CHO-DG44 cells or the derivatives/progenies of any of such cell line. In certain embodiments, the mammalian cells are CHO cells, such as CHO-DG44, CHO-K1 and BHK21, and even more preferred are CHO-DG44 and CHO-K1 cells. In certain embodiments, the mammalian cells are CHO-DG44 cells. Glutamine synthetase (GS)-deficient derivatives of the mammalian cell, particularly of the CHO-DG44 and CHO-K1 cell are also encompassed. In one embodiment, the mammalian cell is a Chinese hamster ovary (CHO) cell, for example a CHO-DG44 cell, a CHO-K1 cell, a CHO DXB11 cell, a CHO-S cell, a CHO GS deficient cell or a derivative thereof.

In certain embodiments, the host cell may further comprise one or more expression cassette(s) encoding a heterologous protein, such as a therapeutic protein, for example a recombinant secreted therapeutic protein. In certain embodiments, the host cells may also be murine cells such as murine myeloma cells, such as NS0 and Sp2/0 cells or the derivatives/progenies of any of such cell line.

The expression of the biologic product of interest or recombinant protein occurs in a cell comprising a DNA sequence coding for the biologic product of interest or recombinant protein, which is transcribed and translated into the protein sequence including post-translational modifications to produce the biologic product of interest or recombination protein in cell culture.

Disclosed herein is a method of manufacturing a biologic product of interest comprising the steps of:
(I) cultivating a eukaryotic cell expressing the biologic product of interest in cell culture;
(II) harvesting the biologic product of interest from the cell culture in the form of a fluid feed comprising biologic product of interest and one or more impurities or buffer components;
(III) purifying the fluid feed comprising biologic product of interest and one or more impurities or buffer components to isolate the biologic product of interest from the fluid feed; and
(IV) optionally formulating the biologic product of interest into a pharmaceutically acceptable formulation suitable for administration; and
wherein the method further comprises the step of: passing the fluid feed through a single-use system for integrated, continuous processing of an initial biologic product;
wherein the single-use system for integrated, continuous processing of an initial biologic product comprises a viral filtration unit operation coupled to a single-pass tangential flow filtration (SPTFF) and diafiltration (DF) unit operation.

Disclosed herein is a method of manufacturing a biologic product of interest comprising the steps of:
(I) cultivating a eukaryotic cell expressing the biologic product of interest in cell culture;
(II) harvesting the biologic product of interest from the cell culture in the form of a fluid feed comprising biologic product of interest and one or more impurities or buffer components;
(III) purifying the fluid feed comprising biologic product of interest and one or more impurities or buffer components to isolate the biologic product of interest from the fluid feed; and
(IV) optionally formulating the biologic product of interest into a pharmaceutically acceptable formulation suitable for administration; and wherein the method further comprises:
a) providing a feed stream (or fluid feed) comprising an initial biologic product; b) filtering the feed stream to remove viral contaminants; c) concentrating the initial biologic product; and d) conducting buffer exchange to produce a processed biologic product.

In certain embodiments, the biologic product of interest is a recombinant protein. In certain embodiments, the step of cultivating a eukaryotic cell expressing the biologic product of interest in cell culture occurs in a fed-batch cell culture. In certain embodiments, wherein the step of cultivating a eukaryotic cell expressing the biologic product of interest in cell culture occurs in a continuous cell culture.

Although the invention has been described with reference to specific embodiments, these descriptions are not meant to be construed in a limiting sense. Various modifications of the disclosed embodiments, as well as alternative embodiments of the invention will become apparent to persons of ordinary skill in the art upon reference to the description of the invention. It should be appreciated by those of ordinary skill in the art that the conception and the specific embodiments disclosed may be readily utilized as a basis for modifying or designing other structures or methods for carrying out the same purposes of the invention. It should also be realized by those of ordinary skill in the art that such equivalent constructions do not depart from the spirit and scope of the invention as set forth in the appended claims. It is therefore, contemplated that the claims will cover any such modifications or embodiments that fall within the scope of the invention.

1-32. (canceled)

33. A single-use system for integrated, continuous processing of an initial biologic product, wherein the system comprises a viral filtration unit operation coupled to a single-pass tangential flow filtration (SPTFF) and diafiltration (DF) unit operation.

34. The single-use system of claim 33, wherein the processing comprises filtration, concentration and buffer exchange.

35. The single-use system of claim 33, wherein the biologic product is a protein and/or a monoclonal antibody.

36. The single-use system of claim 33, wherein the biologic product is a monoclonal antibody.

37. The single-use system of claim 33, wherein the viral filtration unit operation comprises a pump, at least one pre-filter and one or more viral reduction filtration membranes.

38. The single-use system of claim 33, wherein the SPTFF-DF unit operation comprises one or more SPTFF membranes, DF mixing tanks, DF membranes, sensors, pumps or a combination thereof.

39. The single-use system of claim 33, further comprising a feed reservoir coupled to the viral filtration unit operation.

40. The single-use system of claim 39, wherein the feed reservoir holds purified and polished monoclonal antibody at a concentration between about 5 and about 20 g/L.

41. The single-use system of claim 39, wherein the feed reservoir holds purified and polished monoclonal antibody at a concentration of between about 8 and about 12 g/L.

42. The single-use system of claim 33, wherein the system performs integrated, continuous viral filtration, concentration and buffer exchange within a period of about 24 hours or less.

43. The single-use system of claim 33, wherein the system performs integrated, continuous viral filtration, concentration and buffer exchange within a period of about 12 hours or less.

44. The single-use system of claim 33, wherein the system performs integrated, continuous viral filtration, concentration and buffer exchange within a time frame of about 8 hours or less.

45. The single-use system of claim 33, wherein the system performs integrated, continuous viral filtration, concentration and buffer exchange within a time frame of about 8 hours.

46. The single-use system of claim 33, wherein the system is capable of processing a ten-fold increase in the concentration of the biologic product.

47. An integrated, continuous method for providing a processed biologic product, comprising a) providing a feedstream comprising an initial biologic product; b) filtering the feed stream to remove viral contaminants; c) concentrating the initial biologic product; and d) conducting buffer exchange to produce a processed biologic product, wherein the steps b)-d) are carried out by a viral filtration unit operation coupled to a single-pass tangential flow filtration (SPTFF) and diafiltration (DF) unit operation.

48. The method of claim 47, wherein the viral filtration unit operation comprises a pump, at least one pre-filter and one or more viral reduction filtration membranes.

49. The method of claim 47, wherein the SPTFF-DF unit operation comprises one or more SPTFF membranes, DF mixing tanks, DF membranes, sensors, pumps or a combination thereof.

50. A method of manufacturing a biologic product of interest comprising the steps of:
(I) cultivating a eukaryotic cell expressing the biologic product of interest in cell culture;
(II) harvesting the biologic product of interest from the cell culture in the form of a fluid feed comprising biologic product of interest and one or more impurities or buffer components;
(III) purifying the fluid feed comprising biologic product of interest and one or more impurities or buffer components to isolate the biologic product of interest from the fluid feed; and
(IV) optionally formulating the biologic product of interest into a pharmaceutically acceptable formulation suitable for administration;
wherein step IV of the method further comprises the step of: passing the fluid feed through a single-use system for integrated, continuous processing of an initial biologic product;
wherein the single-use system for integrated, continuous processing of an initial biologic product comprises a viral filtration unit operation coupled to a single-pass tangential flow filtration (SPTFF) and diafiltration (DF) unit operation.

51. A method of manufacturing a biologic product of interest comprising the steps of:
(I) cultivating a eukaryotic cell expressing the biologic product of interest in cell culture;
(II) harvesting the biologic product of interest from the cell culture in the form of a fluid feed comprising biologic product of interest and one or more impurities or buffer components;
(III) purifying the fluid feed comprising biologic product of interest and one or more impurities or buffer components to isolate the biologic product of interest from the fluid feed; and
(IV) optionally formulating the biologic product of interest into a pharmaceutically acceptable formulation suitable for administration; and
wherein step IV of the method further comprises:
a) providing a feedstream comprising an initial biologic product; b) filtering the feed stream to remove viral contaminants; c) concentrating the initial biologic product; and
d) conducting buffer exchange to produce a processed biologic product.
